(19) United States
(12) Patent Application Publication
(10) Pub. No.: US 2024/0392034 A1
(43) Pub. Date: Nov. 28, 2024

(54) CLINICAL ASSESSMENT OF M-PROTEIN RESPONSE IN MULTIPLE MYELOMA

(71) Applicant: MORPHOSYS AG, Planegg/Munich (DE)

(72) Inventor: Stefan Härtle, Mammendorf (DE)

(73) Assignee: MORPHOSYS AG, Planegg/Munich (DE)

(21) Appl. No.: 18/802,877

(22) Filed: Aug. 13, 2024

Related U.S. Application Data
(63) Continuation of application No. 18/168,930, filed on Feb. 14, 2023.
Application No. 18/168,930 is a continuation of application No. 16/080,870, filed on Aug. 29, 2018, now Pat. No. 11,618,789.

(30) Foreign Application Priority Data
Mar. 4, 2016 (EP) 16158714.2

(51) Int. Cl. C07K 16/30; A61P 35/00; C07K 14/76; C07K 16/42; G01N 33/49; G01N 33/561; G01N 33/574

(52) U.S. Cl. CPC C07K 16/30; A61P 35/00; C07K 14/76; C07K 16/42; G01N 33/49; G01N 33/561; G01N 33/57426; C07K 2317/56; C07K 2317/565; C07K 2319/31; G01N 2800/52

(57) ABSTRACT

Applicant discloses an anti-idiotypic antibody to MOR202. which when fused to human albumin. shifted the antibody in IFE thus mitigating any potential interference of MOR202 with the M-protein clinical assessment.

### CROSS-REFERENCE TO RELATED APPLICATIONS

The patent application is a continuation of U.S. application Ser. No. 18/168,930 filed Feb. 14, 2023 which is a continuation of U.S. application Ser. No. 16/080,870 filed Aug. 29, 2018, now U.S. Pat. No. 11,618,789, which is the U.S. National Stage of International Application No. PCT/EP2017/055011 filed Mar. 3, 2017, which claims the benefit of EP 16158714.2 filed Mar. 4, 2016, each of which is incorporated by reference in its entirety.

### REFERENCE TO A SEQUENCE LISTING

The contents of the electronic sequence listing (MOR0060USC1.xml); Size 43,615bytes; and Date of Creation: Aug. 13, 2024 is herein incorporated by reference in its entirety.

### BACKGROUND

Multiple myeloma (MM) is a hematological cancer that involves the clonal expansion of malignant plasma cells. MM is the most common malignant plasma cell tumor and the second most common hematologic malignancy in the United States. The US age-adjusted incidence rate is 5.5 cases per 100,000 and the annual incidence reaches approximately 6 to 7 per 100,000 in the United Kingdom.

Plasma cells produce immunoglobulins (also called gammaglobulins), which consist of a heavy chain (IgG, IgA, IgM, IgD or IgE) and a light chain (kappa or lambda) linked together. One plasma cell produces one type of immunoglobulin (for instance, IgA kappa or IgG kappa). Normally the body contains a variety of different plasma cells (“polyclonal”), thus the immunoglobulins in the serum also represent a broad spectrum of different formats and specificities (polyclonal). In the case of multiple myeloma, the malignant cells are copies of only one or only a few distinct plasma cell(s) and the Immunoglobulin secreted by this or these cell(s) is considered as monoclonal.

This monoclonal immunoglobulin is called M-protein or paraprotein and may also consist of a heavy chain (most often IgG or IgA but also IgM, IgD or IgE) and a light chain (kappa or lambda) or truncated forms of these immunoglobulins. The increase of M protein in the serum is used to identify B-cell malignancies, such as, MM.

Multiple staging systems are currently used for the diagnosis and monitoring of responses in multiple myeloma: a) the Durie and Salmon Staging System, b) the International Staging System (ISS), and the International Myeloma Working Group (IMWG). The Durie and Salmon staging system involves features that assess tumour cells mass, elevated serum immunoglobulin (Ig)G levels, end-organ damage, and osteolytic bone lesions. The ISS places more emphasis on the disease burden based on β2-microglobulin levels and serum albumin levels. The IMWG takes into account both molecular and cytogenetic abnormalities, specifically, M-protein reduction over time is one of the most important factors and is used to assess the progress of disease and treatment success.

Protein manifestations characteristic of multiple myeloma include increases of monoclonal (M)-protein concentrations (IgG, IgA, IgA, IgD), light chain concentrations (including kappa [κ] and lambda [λ]), abnormal β2-microglobulin, serum albumin, creatinine, and hemoglobin levels, and findings of bone marrow plasma cells (of greater than or equal to 5%). Measurement of the protein manifestations (such as M protein) produced by patients can be achieved by numerous methods. Tests that measure M-proteins are the 24-hour urine collection test, urine protein electrophoresis (UPEP), serum protein electrophoresis (SPEP), immunofixation electrophoresis (IFE), and serum free light chain (sFLC) assay.

CD38 is an example of an antigen expressed on malignant plasma cells, and other lymphocytes, and therefore, represents a therapeutic target in the treatment of multiple myeloma and other gammopathies. Functions ascribed to CD38 include both receptor mediation in adhesion and signaling events and (ecto-) enzymatic activity. As an ectoenzyme, CD38 uses NAD+ as substrate for the formation of cyclic ADP-ribose (cADPR) and ADPR, but also of nicotinamide and nicotinic acid-adenine dinucleotide phosphate (NAADP). cADPR and NAADP have been shown to act as second messengers for Ca2+ mobilization. By converting NAD+ to cADPR, CD38 regulates the extracellular NAD+ concentration and hence cell survival by modulation of NAD-induced cell death (NCID). In addition to signaling via Ca2+, CD38 signaling occurs via cross-talk with antigen-receptor complexes on T and B cells or other types of receptor complexes, e.g. MHC molecules, and is in this way involved in several cellular responses, but also in switching and secretion of IgG.

Antibodies specific for CD38 are in development for the treatment of multiple myeloma. Antibodies specific for CD38 are described in WO1999/62526 (Mayo Foundation); WO200206347 (Crucell Holland); US2002164788 (Jonathan Ellis) which is incorporated by reference in its entirety; WO2005/103083 (MorphoSys AG), U.S. Ser. No. 10/588,568, which is incorporated by reference in its entirety, WO2006/125640 (MorphoSys AG), U.S. Ser. No. 11/920,830, which is incorporated by reference in its entirety, and WO2007/042309 (MorphoSys AG), U.S. Ser. No. 12/089,806, which is incorporated by reference in its entirety; WO2006099875 (Genmab), U.S. Ser. No. 11/886,932, which is incorporated by reference in its entirety; WO2011154453A1 (Genmab), U.S. Ser. No. 13/702,857, which is incorporated by reference in its entirety; WO08/047242 (Sanofi-Aventis), U.S. Ser. No. 12/441,466, which is incorporated by reference in its entirety; WO2015066450 (Sanofi), U.S. Ser. No. 14/529,719, which is incorporated by reference in its entirety; WO2012092616A1, and WO2012092612A1(Takeda), U.S. Ser. Nos. 13/341,860, and 13/977,207 both of which are incorporated by reference in their entirety, and WO2014178820A1 (Teva).

Anti-CD38 antibody treatment in MM patients can result in partial or complete clearance of the M-protein produced by multiple myeloma cells. Serum protein electrophoresis (SPEP) and immunofixation electrophoresis (IFE) are both essential assays used for identifying and immunotyping monoclonal proteins in patients with multiple myeloma. Recent studies demonstrated that certain therapeutic antibodies in development for the treatment of multiple myeloma are readily detected on serum IFE and can interfere with the detection and monitoring of M protein levels (McCudden et al., Clinical Chemistry, 56:12; 1897-1904 (2010), see also Genzen et al., British Journal of Haematology (2011) 155(1) 123-125). McCudden et al. observed that an incubation with Siltuximab (an anti-IL-6 antibody) with anti-drug antibodies shifted the drug electrophoretic pattern such that the therapeutic antibody Siltuximab could be differentiated from endogenous M-protein. Janssen also recently published the development of a clinical assay to mitigate Daratumumab's interference with M-protein in IFE using a similar approach, which utilized a mouse anti-daratumumab antibody ideally labeled with biotin or Alexa-fluor tags to shift the complex on IFE. Axel, et al., Development of a Clinical Assay to Mitigate Daratumumab, an IgG1k Monoclonal Antibody, Interference with Serum Immunofixation and Clinical Assessment of M-protein Response in Multiple Myeloma Poster Presented at the 105^{th} Annual Meeting of the American Association for Cancer Research (AACR), Apr. 5-9, 2014, San Diego, California, USA; see also van de Donk et al., Monoclonal antibodies targeting CD38 in hematological malignancies and beyond, Immunological Reviews, 270:95-112 (2016).

These approaches, however, are not sufficient for every therapeutic antibody. Novel mitigation strategies are needed that are specific for each therapeutic antibody to avoid this potential interference with SPEP and IFE to ensure valid clinical response descriptions that meet the International Myeloma Working Group (IMWG) criteria.

### SUMMARY OF THE INVENTION

Applicant herein discloses an anti-idiotypic antibody to MOR202, which when fused to human albumin, shifted the antibody in IFE thus mitigating any potential interference of MOR202 with the M-protein based clinical assessment.

The anti-idiotypic antibody, albumin fusion will be integrated into the clinical development design of MOR202 in order to enhance the clinical assessment of the M-protein response.

An aspect is an anti-idiotypic antibody to MOR202. In an aspect the anti-idiotypic antibody is fused to human albumin. In embodiments the anti-idiotypic antibody comprises a variable heavy chain comprising
an HCDR1 of the amino acid sequence YSFSNYWIS (SEQ ID NO: 18),
an HCDR2 of the amino acid sequence WMGIIDPASSKTRYSPSFQG (SEQ ID NO: 19),
an HCDR3 of the amino acid sequence SRGAGMDY (SEQ ID NO: 20), and
a variable light chain comprising an LCDR1 of the amino acid sequence TGSSSNIGAGYDVH (SEQ ID NO: 21),
an LCDR2 of the amino acid sequence LLIYADNNRPS (SEQ ID NO: 22),
an LCDR3 of the amino acid sequence GSYDESSNSM (SEQ ID NO: 23).

In an embodiment, the anti-idiotypic antibody is a human antibody.

In embodiments, the anti-idiotypic antibody fusion comprises the heavy chain of amino acid sequence

(SEQ ID NO: 24)
QVQLVQSGAEVKKPGESLKISCKGSGYSFSNYWISWVRQMPGKGLEWMGI
IDPASSKTRYSPSFQGQVTISADKSISTAYLQWSSLKASDTAMYYCARSR
GAGMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSDIDAHKSEVAHRFKDLGEENFKALVLIAFAQ
YLQQCPFEDHVKLVNEVTEFAKTCVADESAENCDKSLHTLFGDKLCTVAT
LRETYGEMADCCAKQEPERNECFLQHKDDNPNLPRLVRPEVDVMCTAFHD
NEETFLKKYLYEIARRHPYFYAPELLFFAKRYKAAFTECCQAADKAACLL
PKLDELRDEGKASSAKQRLKCASLQKFGERAFKAWAVARLSQRFPKAEFA
EVSKLVTDLTKVHTECCHGDLLECADDRADLAKYICENQDSISSKLKECC
EKPLLEKSHCIAEVENDEMPADLPSLAADFVESKDVCKNYAEAKDVFLGM
FLYEYARRHPDYSVVLLLRLAKTYETTLEKCCAAADPHECYAKVFDEFKP
LVEEPQNLIKQNCELFEQLGEYKFQNALLVRYTKKVPQVSTPTLVEVSRN
LGKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTAL
VELVKHKPKATKEQLKAVMDDFAAFVEKCCKADDKETCFAEEGKKLVAAS
QAALGLVNSRHHHHHH.

In embodiments, the anti-idiotypic antibody fusion comprises the light chain of amino acid sequence

(SEQ ID NO: 25)
DIVLTQPPSVSGAPGQRVTISCTGSSSNIGAGYDVHWYQQLPGTAPKLLI
YADNNRPSGVPDRFSGSKSGTSASLAITGLQSEDEADYYCGSYDESSNSM
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS

An aspect is a method of evaluating a blood sample obtained from a patient undergoing treatment for multiple myeloma or other gammopathy comprising
obtaining a blood sample from said patient,
incubating the blood sample with an anti-idiotypic antibody,
performing immunofixation electrophoresis (IFE), and
reporting the results of the IFE.

In embodiments, the patient is undergoing treatment with MOR202.

In embodiments, the sample is evaluated for total M-protein levels.

An aspect, is a nucleic acid encoding the exemplified anti-idiotypic antibody or exemplified anti-idiotypic antibody albumin fusion.

### BRIEF DESCRIPTION OF DRAWINGS

FIG. 1 shows the amino acid sequence of MOR202.

FIGS. 2A-B show the amino acid sequence of MOR09292 (an anti-idiotypic antibody to MOR202) human albumin fusion protein.

FIG. 3 shows a typical normal pattern for the distribution of proteins as determined by serum protein electrophoresis.

FIG. 4 shows a serum protein electrophoresis distribution of proteins having a homogeneous spike-like peak in a focal region of the gamma-globulin zone common to disorders known as monoclonal gammopathies. This peak represents a single clone of plasma cells that produce a homogeneous M protein.

FIG. 5 shows an example of a gel after serum immunofixation electrophoresis of a healthy donor. Lane ELP=total protein staining; Lane G=anti-IgG staining; Lane A=anti-IgA staining; Lane M=anti-IgM staining; Lane K=anti-kappa staining; Lane L=anti-lambda staining.

FIG. 6 shows a serum immunofixation electrophoresis of a sample from a drug naïve healthy donor (A and B) and a drug naïve MM patient (C and D). The samples were tested either unspiked or spiked with MOR202 at different concentrations. (Lane 1=no MOR202 added; Lane 2=MOR202 added at 200 μg/mL; Lane 3=MOR202 added at 400 μg/mL; Lane 4=MOR202 added at 800 μg/mL; Lane 5=MOR202 added at 1200 μg/mL). Bands encircled with a dotted line become only visible after MOR202 spike and represent the respective molecule. Bands marked with an arrow represent endogenous M-protein.

FIG. 7 shows a serum immunofixation electrophoresis of MOR202 +/− preincubation of MOR0929 IgG1 and MOR09292 IgM in saline. MOR202 at a constant concentration of 1200 μg/mL (A and B) or 560 μg/mL (C) in saline was pre-incubated with its idiotypic antibody MOR09292 in different formats and the prepared samples were analyzed via IFE. A)+B): MOR202 and MOR09292 IgG1 (using anti-IgG staining (A) and anti-lambda staining (B) (Lane 1=MOR202; Lane 2=MOR09292 IgG1 2400 μg/mL; Lane 3=MOR202+MOR09292 IgG1 at 600 μg/mL; Lane 4=MOR202+MOR09292 IgG1 at 1200 μg/mL; Lane 5=MOR202+MOR09292 IgG1 at 2400 μg/mL). C): MOR202 and MOR09292 IgM using anti-IgG staining (Lane 2-4), anti-lambda staining (Lane 5-7) and anti-IgM staining (Lane 8-10) (Lane 2/5/8=MOR202; Lane 3/6/9=MOR09292 IgM 560 μg/mL; Lane 4/7/10=MOR202+MOR09292 IgM 560 μg/mL; Lane 1=human serum from a healthy donor stained for total protein to evaluate general background signaling in serum samples).

FIG. 8 shows a serum immunofixation electrophoresis of MOR202 +/− preincubation of MOR09292-human albumin fusion (MOR09292-hAlb) in saline and human serum. MOR202 at a constant concentration of 1200 μg/mL in saline (Lane 2-3) or serum (Lane 4-13) was pre-incubated with or without its idiotypic antibody MOR09292-hAlb at different ratios and the prepared samples were analyzed via IFE using anti-IgG staining (Lane 2-8) or anti-lambda staining (Lane 9-13). (Lane 2=MOR202; Lane 3=MOR202+MOR09292-hAlb at 2400 μg/mL; Lane 4=MOR202; Lane 5=MOR202+MOR09292-hAlb at 1200 μg/mL; Lane 6=MOR202+MOR09292-hAlb at 1800 μg/mL; Lane 7=MOR202+MOR09292-hAlb at 2400 μg/mL; Lane 8=MOR202+MOR09292-hAlb at 3600 μg/mL; Lane 9=MOR202; Lane 10=MOR202+MOR09292-hAlb at 1200 μg/mL; Lane 11=MOR202+MOR09292-hAlb at 1800 μg/mL; Lane 12=MOR202+MOR09292-hAlb at 2400 μg/mL; Lane 13=MOR202+MOR09292-hAlb at 3600 μg/mL; Lane 1=human serum from a healthy donor stained for total protein to evaluate general background signaling).

### DETAILED DESCRIPTION OF THE INVENTION

### Definitions

The term “anti-idiotypic” describes a protein or peptide that binds to the variable regions of an antibody. The anti-idiotypic protein can be an antibody. For example, antibody MOR09292 binds to the variable regions of MOR202.

The term “antibody” includes antibody fragments. Antibodies include monoclonal antibodies of any isotype, e.g., IgG, IgM, IgA, IgD and IgE. An IgG antibody is comprised of two identical heavy chains and two identical light chains that are joined by disulfide bonds. The heavy and light chains of antibodies contain a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′ F (ab′)2 fragment, or other fragment, which contains at least one variable heavy or variable light chain, each containing CDRs and framework regions.

The “CDRs” herein are defined by either Chothia et al., Kabat et al. or by an internal numbering convention. See Chothia C, Lesk A M. (1987) Canonical structures for the hypervariable regions of immunoglobulins. J Mol Biol., 196(4):901-17, which is incorporated by reference in its entirety. See Kabat E. A, Wu T. T., Perry H. M., Gottesman K. S. and Foeller C. (1991). Sequences of Proteins of Immunological Interest. 5th edit., NIH Publication no. 91-3242, US Dept. of Health and Human Services, Washington, DC, which is incorporated by reference in its entirety.

“VH” refers to the variable region of an immunoglobulin heavy chain of an antibody, or antibody fragment. “VL” refers to the variable region of the immunoglobulin light chain of an antibody, or antibody fragment.

“Fc region” means the constant region of an antibody, which in humans may be of the IgG1, 2, 3, 4 subclass or others. The sequences of human Fc regions are available at IMGT, Human IGH C-REGIONs, http with the extension imgt.org/ligmdb/of the world wide web (retrieved on 22 Feb. 2016).

A “human antibody” or “human antibody fragment”, as used herein, includes antibodies and antibody fragments having variable regions in which both the framework and CDR regions are derived from sequences of human origin. Furthermore, if the antibody contains a constant region, the constant region also is derived from such sequences.

“Specific” describes a protein that recognizes an antigen and is able to discriminate between such antigen and one or more reference antigen(s). This ability can be identified by a standard ELISA assay. Typically, determination of specificity is performed by using not a single reference antigen, but a set of about three to five unrelated antigens, such as milk powder, BSA, transferrin or the like.

“Evaluating a blood sample” means evaluating the blood or portion of the blood sample most relevant for the method. Currently immunofixation electrophoresis is done on the serum component of blood. If, however, in the future a different blood component is evaluated, the invention is directed to a method evaluating that blood component. Blood components include, for example, plasma, serum, cells, e.g. red and white cells, and platelets. Plasma includes proteins, such as globulins, and clotting factors, and salts, sugars, fat, hormones and vitamins.

Gammopathies are conditions in which serum immunoglobulin levels are greatly increased. They can be classified either as polyclonal (increases in all major immunoglobulin classes) or monoclonal (increases in a single homogeneous immunoglobulin).

Polyclonal gammopathies result from chronic stimulation of the immune system. They can therefore be caused by chronic pyodermas; chronic viral, bacterial, or fungal infections; granulomatous bacterial diseases; abscesses; chronic parasitic infections; chronic rickettsial diseases, such as tropical canine pancytopenia; chronic immunologic diseases, such as systemic lupus erythematosus, rheumatoid arthritis, and myositis; or by some neoplasia. In many cases, there is no obvious predisposing cause. In some animals, the gammopathy may initially be monoclonal because of the predominance of a single immunoglobulin class (usually IgG).

Monoclonal gammopathies are characterized by the production of large amounts of a single immunoglobulin protein. Monoclonal gammopathies are either benign (ie, associated with no underlying disease), or more commonly, associated with immunoglobulin-secreting tumors. Tumors that secrete monoclonal antibodies originate from plasma cells (myelomas). Myelomas can secrete intact proteins of any immunoglobulin class or immunoglobulin subunits or fragments (light chains or heavy chains). Examples of Monoclonal gammopathies include: Hodgkin's disease; variants of multiple myeloma, e.g., Solitary plasmacytoma of bone, Extramedullary plasmacytoma, Plasma cell leukemia, and Non-secretory myeloma, Lymphoproliferative disorders, such as, Waldenström's macroglobulinemia, and Lymphoma; Heavy chain disease (γ, α, μ); and Amyloidosis.

The term “CD38” refers to the protein known as CD38, having the following synonyms: ADP-ribosyl cyclase 1, cADPr hydrolase 1, Cyclic ADP-ribose hydrolase 1, T10.

Human CD38 has the amino acid sequence of:

(SEQ ID NO: 1)
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTS
EI.

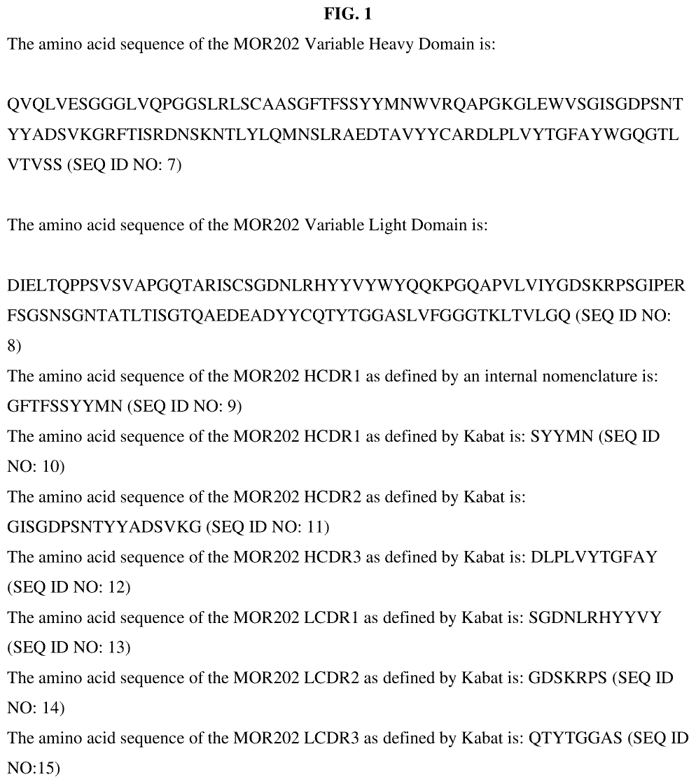

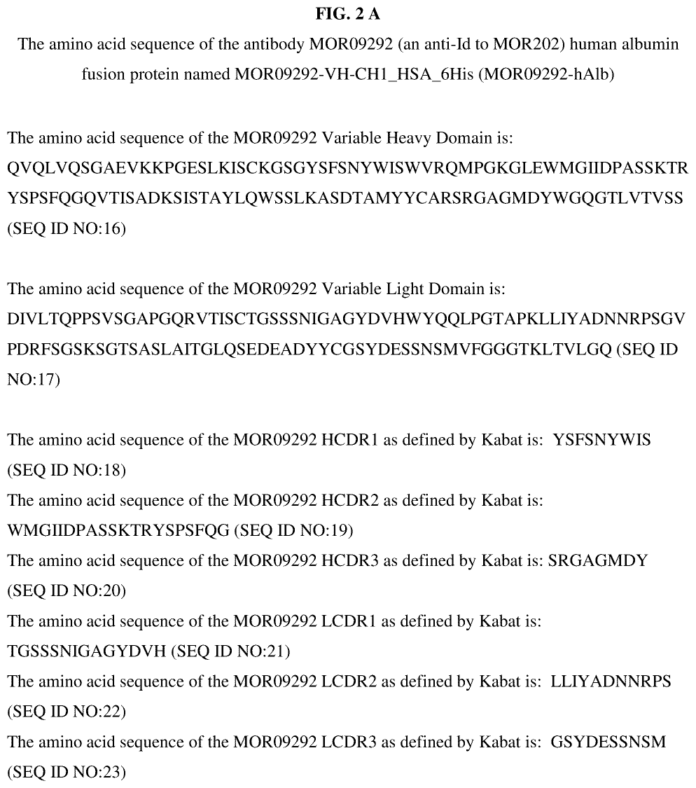

“MOR202” an anti-CD38 antibody whose amino acid sequence is provided in FIG. 1. “MOR202” and “MOR03087” are used as synonyms to describe the antibody shown in FIG. 1.

The DNA sequence encoding the MOR202 Variable Heavy Domain is:

(SEQ ID NO: 2)
CAGGTGCAATTGGTGGAAAGCGGCGGCGGCCTGGTGCAACCGGGCGGCAG
CCTGCGTCTGAGCTGCGCGGCCTCCGGATTTACCTTTTCTTCTTATTATA
TGAATTGGGTGCGCCAAGCCCCTGGGAAGGGTCTCGAGTGGGTGAGCGGT
ATCTCTGGTGATCCTAGCAATACCTATTATGCGGATAGCGTGAAAGGCCG
TTTTACCATTTCACGTGATAATTCGAAAAACACCCTGTATCTGCAAATGA
ACAGCCTGCGTGCGGAAGATACGGCCGTGTATTATTGCGCGCGTGATCTT
CCTCTTGTTTATACTGGTTTTGCTTATTGGGGCCAAGGCACCCTGGTGAC
GGTTAGCTCA

The DNA sequence encoding the MOR202 Variable Light Domain is:

(SEQ ID NO: 3)
GATATCGAACTGACCCAGCCGCCTTCAGTGAGCGTTGCACCAGGTCAGAC
CGCGCGTATCTCGTGTAGCGGCGATAATCTTCGTCATTATTATGTTTATT
GGTACCAGCAGAAACCCGGGCAGGCGCCAGTTCTTGTGATTTATGGTGAT
TCTAAGCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTGCCAGACTTATACTGGTGGTGCTTCTCTTGTGTTTGGCGGC
GGCACGAAGTTAACCGTTCTTGGCCAG

MOR202 is disclosed in WO2007/042309, U.S. Ser. No. 12/089,806, which is incorporated by reference in its entirety. In U.S. Ser. No. 12/089,806, MOR202 is the antibody comprising the variable heavy chain corresponding to SEQ ID NO: 21 and variable light chain corresponding to SEQ ID NO: 51, and the nucleic acids encoding MOR202 are variable heavy chain SEQ ID NO: 6 and variable light chain SEQ ID NO:36.

MOR202 is currently being tested in a phase 1/2a trial in patients with relapsed/refractory myeloma. The study is evaluating the safety and preliminary efficacy of MOR202 as monotherapy and in combination with pomalidomide and lenalidomide plus dexamethasone.

The antibody, MOR09292, is an anti-idiotypic antibody to MOR202 and is encoded by the nucleic acid sequence:

VH:
(SEQ ID NO: 4)
CAGGTGCAATTGGTTCAGAGCGGCGCGGAAGTGAAAAAACCGGGCGAAAG
CCTGAAAATTAGCTGCAAAGGTTCCGGATATTCCTTTTCTAATTATTGGA
TTTCTTGGGTGCGCCAGATGCCTGGGAAGGGTCTCGAGTGGATGGGCATT
ATCGATCCGGCTTCTAGCAAGACCCGTTATTCTCCGAGCTTTCAGGGCCA
GGTGACCATTAGCGCGGATAAAAGCATTAGCACCGCGTATCTTCAATGGA
GCAGCCTGAAAGCGAGCGATACGGCCATGTATTATTGCGCGCGTTCTCGT
GGTGCTGGTATGGATTATTGGGGCCAAGGCACCCTGGTGACGGTTAGCTC
A
VL:
(SEQ ID NO: 5)
GATATCGTGCTGACCCAGCCGCCTTCAGTGAGTGGCGCACCAGGTCAGCG
TGTGACCATCTCGTGTACGGGCAGCAGCAGCAACATTGGTGCTGGTTATG
ATGTGCATTGGTACCAGCAGTTGCCCGGGACGGCGCCGAAACTTCTGATT
TATGCTGATAATAATCGTCCCTCAGGCGTGCCGGATCGTTTTAGCGGATC
CAAAAGCGGCACCAGCGCGAGCCTTGCGATTACGGGCCTGCAAAGCGAAG
ACGAAGCGGATTATTATTGCGGTTCTTATGATGAGTCTTCTAATTCTATG
GTGTTTGGCGGCGGCACGAAGTTAACCGTTCTTGGCCAG

DNA encoding MOR09292-VH-CH1_HSA_6His (no leader sequence) (MOR09292-hAlb heavy chain):

(SEQ ID NO: 26)
CAGGTGCAATTGGTTCAGAGCGGCGCGGAAGTGAAAAAACCGGGCGAAAG
CCTGAAAATTAGCTGCAAAGGTTCCGGATATTCCTTTTCTAATTATTGGA
TTTCTTGGGTGCGCCAGATGCCTGGGAAGGGTCTCGAGTGGATGGGCATT
ATCGATCCGGCTTCTAGCAAGACCCGTTATTCTCCGAGCTTTCAGGGCCA
GGTGACCATTAGCGCGGATAAAAGCATTAGCACCGCGTATCTTCAATGGA
GCAGCCTGAAAGCGAGCGATACGGCCATGTATTATTGCGCGCGTTCTCGT
GGTGCTGGTATGGATTATTGGGGCCAAGGCACCCTGGTGACGGTTAGCTC
AGCCTCCACCAAGGGTCCATCGGTCTTCCCCCTGGCACCCTCCTCCAAGA
GCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCA
GCGTGGTGACCGTGCCCTCCAGCAGCTTGGGCACCCAGACCTACATCTGC
AACGTGAATCACAAGCCCAGCAACACCAAGGTGGACAAGAGAGTTGAGCC
CAAATCTGATATCGACGCCCACAAGAGCGAGGTGGCCCACCGGTTTAAGG
ACCTGGGCGAGGAAAACTTCAAGGCCCTGGTGCTGATCGCCTTCGCCCAG
TACCTGCAGCAGTGCCCCTTCGAGGACCACGTGAAGCTCGTGAACGAAGT
GACCGAGTTCGCCAAGACCTGCGTGGCCGATGAGAGCGCCGAGAACTGCG
ACAAGAGCCTGCACACCCTGTTCGGCGACAAGCTGTGTACCGTGGCCACC
CTGAGAGAAACCTACGGCGAGATGGCCGACTGCTGCGCCAAGCAGGAACC
CGAGAGGAACGAGTGCTTCCTGCAGCACAAGGACGACAACCCCAACCTGC
CCAGACTCGTGCGGCCCGAAGTGGACGTGATGTGCACCGCCTTCCACGAC
AACGAGGAAACCTTCCTGAAGAAGTACCTGTACGAGATCGCCAGACGGCA
CCCCTACTTCTACGCCCCCGAGCTGCTGTTCTTCGCCAAGCGGTACAAGG
CCGCCTTCACCGAGTGTTGCCAGGCCGCCGATAAGGCCGCTTGCCTGCTG
CCTAAGCTGGACGAGCTGAGGGATGAGGGCAAGGCCAGCTCTGCCAAGCA
GAGACTGAAGTGCGCCAGCCTGCAGAAGTTCGGCGAGCGGGCCTTTAAAG
CCTGGGCCGTGGCTAGACTGAGCCAGAGATTCCCCAAGGCCGAGTTTGCC
GAGGTGTCCAAGCTCGTGACCGACCTGACCAAGGTGCACACCGAGTGCTG
TCACGGCGACCTGCTGGAATGCGCCGACGACAGAGCCGATCTGGCCAAGT
ACATCTGCGAGAACCAGGACAGCATCAGCAGCAAGCTGAAAGAGTGCTGC
GAGAAGCCTCTGCTGGAAAAGAGCCACTGTATCGCCGAGGTGGAAAACGA
CGAGATGCCCGCCGATCTGCCTTCTCTGGCCGCCGACTTCGTGGAAAGCA
AGGACGTGTGCAAGAACTACGCCGAGGCCAAGGATGTGTTCCTGGGCATG
TTTCTGTATGAGTACGCCCGCAGACACCCCGACTACAGCGTGGTGCTGCT
GCTGAGACTGGCCAAAACCTACGAGACAACCCTGGAAAAGTGCTGTGCCG
CCGCTGACCCCCACGAGTGTTACGCCAAGGTGTTCGACGAGTTCAAGCCA
CTGGTGGAAGAACCCCAGAACCTGATCAAGCAGAATTGCGAGCTGTTCGA
GCAGCTGGGCGAGTACAAGTTCCAGAACGCCCTGCTCGTGCGGTACACCA
AGAAAGTGCCCCAGGTGTCCACCCCCACCCTGGTGGAAGTGTCCCGGAAC
CTGGGCAAAGTGGGCAGCAAGTGCTGCAAGCACCCTGAGGCCAAGAGAAT
GCCCTGCGCCGAGGACTACCTGTCTGTGGTGCTGAACCAGCTGTGCGTGC
TGCACGAGAAAACCCCCGTGTCCGACAGAGTGACCAAGTGCTGTACCGAG
AGCCTCGTGAACAGACGGCCCTGCTTCAGCGCCCTGGAAGTGGATGAGAC
ATACGTGCCCAAAGAGTTCAACGCCGAGACATTCACCTTCCACGCCGACA
TCTGCACCCTGTCCGAGAAAGAGCGGCAGATCAAGAAACAGACCGCTCTG
GTGGAACTCGTGAAGCACAAGCCCAAGGCCACCAAAGAACAGCTGAAGGC
CGTGATGGACGACTTCGCCGCCTTTGTGGAAAAATGCTGCAAGGCCGATG
ACAAAGAGACATGCTTCGCCGAAGAGGGCAAGAAACTGGTGGCCGCCTCT
CAGGCTGCTCTGGGACTGGTTAACTCTAGACACCATCACCATCACCAT.

DNA encoding MOR09292-VL-lambda (no leader sequence) (MOR09292-hAlb light chain):

(SEQ ID NO: 27)
GATATCGTGCTGACCCAGCCGCCTTCAGTGAGTGGCGCACCAGGTCAGCG
TGTGACCATCTCGTGTACGGGCAGCAGCAGCAACATTGGTGCTGGTTATG
ATGTGCATTGGTACCAGCAGTTGCCCGGGACGGCGCCGAAACTTCTGATT
TATGCTGATAATAATCGTCCCTCAGGCGTGCCGGATCGTTTTAGCGGATC
CAAAAGCGGCACCAGCGCGAGCCTTGCGATTACGGGCCTGCAAAGCGAAG
ACGAAGCGGATTATTATTGCGGTTCTTATGATGAGTCTTCTAATTCTATG
GTGTTTGGCGGCGGCACGAAGTTAACCGTCCTAGGTCAGCCCAAGGCTGC
CCCCTCGGTCACTCTGTTCCCGCCCTCCTCTGAGGAGCTTCAAGCCAACA
AGGCCACACTGGTGTGTCTCATAAGTGACTTCTACCCGGGAGCCGTGACA
GTGGCCTGGAAGGCAGATAGCAGCCCCGTCAAGGCGGGAGTGGAGACCAC
CACACCCTCCAAACAAAGCAACAACAAGTACGCGGCCAGCAGCTATCTGA
GCCTGACGCCTGAGCAGTGGAAGTCCCACAGAAGCTACAGCTGCCAGGTC
ACGCATGAAGGGAGCACCGTGGAGAAGACAGTGGCCCCTACAGAATGTTC
A.

Human albumin has the following amino acid sequence (including the signal sequence):

(SEQ ID NO: 6)
MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL.

The International Myeloma Working Group (IMWG) Uniform Response Criteria for Multiple Myeloma are as follows:

| Response | IMWG criteria |
|---|---|
| sCR | CR as defined below plus normal FLC ratio |
| | and absence of clonal cells in bone |
| | marrow by immunohistochemistry or |
| | immunofluorescence |
| CR | Negative immunofixation on the serum and |
| | urine and disappearance of any soft |
| | tissue plasmacytomas and <5% plasma |
| | cells in bone marrow |
| VGPR | Serum and urine M-protein detectable by |
| | immunofixation but not on electrophoresis |
| | or ≥90% reduction in serum M-protein plus |
| | urine M-protein level <100 mg/24 h |
| PR | ≥50% reduction of serum M-protein and |
| | reduction in 24 hours urinary M-protein by |
| | ≥90% or to <200 mg/24 h |
| | If the serum and urine M-protein are |
| | unmeasurable, 5a ≥50% decrease in the |
| | difference between involved and uninvolved |
| | FLC levels is required in place of the |
| | M-protein criteria |
| | If serum and urine M-protein are not |
| | measurable, and serum free light assay is |
| | also |
| | not measureable, ≥50% reduction in |
| | plasma cells is required in place of M- |
| | protein, |
| | provided baseline bone marrow plasma cell |
| | percentage was ≥30% |
| | In addition to the above listed criteria, if |
| | present at baseline, a ≥50% reduction in |
| | the size of soft tissue plasmacytomas is |
| | also required |
| Stable Disease | Not meeting criteria for CR, VGPR, PR, or |
| | progressive disease |
| Progressive Disease | Increase of ≥25% from lowest response |
| | value in any one or more of the following: |
| | Serum M-component and/or (the absolute |
| | increase must be ≥0.5 g/dL)6 |
| | Urine M-component and/or (the absolute |
| | increase must be ≥200 mg/24 h) |
| | Only in patients without measurable serum |
| | and urine M-protein levels; the |
| | difference between involved and uninvolved |
| | FLC levels. The absolute increase |
| | must be >10 mg/dL |
| | Bone marrow plasma cell percentage; the |
| | absolute percentage must be ≥10% 7 |
| | Definite development of new bone lesions |
| | or soft tissue plasmacytomas or |
| | definite increase in the size of existing bone |
| | lesions or soft tissue plasmacytomas |
| | Development of hypercalcaemia (corrected |
| | serum calcium >11.5 mg/dL or 2.65 |
| | mmol/L) that can be attributed solely to the |
| | plasma cell proliferative disorder |
| Relapse | Clinical relapse requires one or more of: |
| | Direct indicators of increasing disease |
| | and/or end organ dysfunction (CRAB |
| | features). It is not used in calculation |
| | of time to progression or progression-free |
| | survival but is listed here as something |
| | that can be reported optionally or for |
| | use in clinical practice |
| | 1. Development of new soft tissue |
| | plasmacytomas or bone lesions |
| | 2. Definite increase in the size of existing |
| | plasmacytomas or bone lesions. A definite |
| | increase is defined as a 50% (and at least 1 |
| | cm) increase as measured serially by |
| | the sum of the products of the cross- |
| | diameters of the measurable lesion |
| | 3. Hypercalcemia (>11.5 mg/dL) [2.65 |
| | mmol/L] |
| | 4. Decrease in haemoglobin of ≥2 g/dL |
| | [1.25 mmol/L] |
| | 5. Rise in serum creatinine by 2 mg/dL or |
| | more [177 mol/L or more] |

Electrophoresis is a method of separating proteins based on their biochemical properties. Serum is placed on a specific medium, and a charge is applied. The net charge (positive or negative) and the size and shape of the protein commonly are used in differentiating various serum proteins.

Several subsets of serum protein electrophoresis are available. The names of these subsets are based on the method that is used to separate and differentiate the various serum components. In zone electrophoresis, for example, different protein subtypes which are placed in separate physical locations on a gel made from agar, cellulose, or other plant material. The proteins are stained, and their densities are calculated electronically to provide graphical data on the absolute and relative amounts of the various proteins. Further separation of protein subtypes is achieved by staining with an immunologically active agent, which results in immunofixation and/or immunofluorescence.

The pattern of serum protein electrophoresis results depends on the fractions of two major types of proteins: albumin and globulins. Albumin, the major protein component of serum, is produced by the liver under normal physiologic conditions. Globulins comprise a smaller fraction of the total serum protein content. The subsets of these proteins and their relative quantity are mostly the primary focus of the interpretation of serum protein electrophoresis.

Albumin, the largest peak observed in serum protein electrophoresis, is located closest to the positive electrode. The next five components (globulins) are labeled alpha1, alpha2, beta1, beta2, and gamma. The peaks for these components appear toward the negative electrode, with the gamma peak being closest to that electrode.

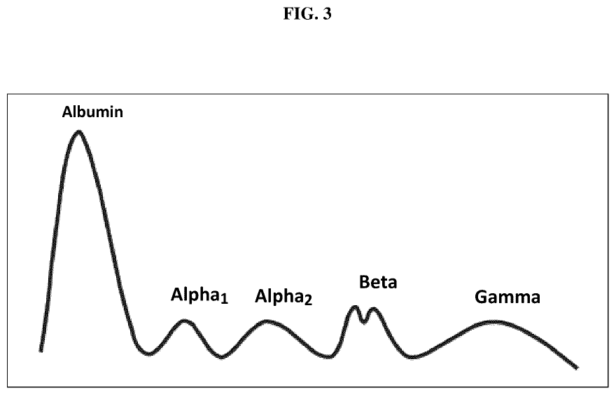

FIG. 3 shows a typical normal pattern for the distribution of proteins as determined by serum protein electrophoresis.

The albumin band represents the largest protein component of human serum. The albumin level is decreased under circumstances in which there is less production of the protein by the liver or in which there is increased loss or degradation of this protein. Malnutrition, significant liver disease, renal loss (e.g., in nephrotic syndrome), hormone therapy, and pregnancy may account for a low albumin level. Burns also may result in a low albumin level. Levels of albumin are increased for example in patients with a relative reduction in serum water (e.g., dehydration).

Moving toward the negative portion of the gel (i.e., the negative electrode), the next peaks involve the alpha1 and alpha2 components. The alpha1-protein fraction is comprised of alpha1-antitrypsin, thyroid-binding globulin, and transcortin. Malignancy and acute inflammation (resulting from acute-phase reactants) can increase the alpha1-protein band. A decreased alpha1-protein band may occur because of alpha1-antitrypsin deficiency or decreased production of the globulin as a result of liver disease. Ceruloplasmin, alpha2-macroglobulin, and haptoglobin contribute to the alpha2-protein band. The alpha2 component is increased as an acute-phase reactant.

The beta fraction has two peaks labeled beta1 and beta2. Beta1 is composed mostly of transferrin, and beta2 contains beta-lipoprotein. IgA, IgM, and sometimes IgG, along with complement proteins, also can be identified in the beta fraction.

Much of the clinical interest is focused on the gamma region of the serum protein spectrum because immunoglobulins migrate to this region. It should be noted that immunoglobulins often can be found throughout the electrophoretic spectrum. C-reactive protein (CRP) is located in the area between the beta and gamma components.

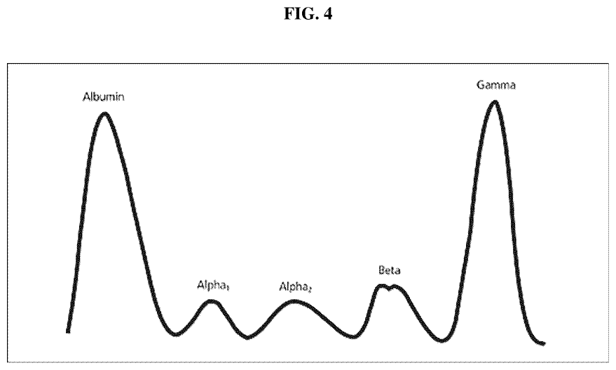

Although many conditions can cause an increase in the gamma region, several disease states cause a homogeneous spike-like peak in a focal region of the gamma-globulin zone (FIG. 4). These so-called “monoclonal gammopathies” constitute a group of disorders that are characterized by proliferation of a single or very few clone(s) of plasma cells each producing a homogeneous M protein, such as MM.

Immunofixation electrophoresis (IFE) is a technique that allows a protein to be anchored after electrophoresis by forming an insoluble complex with a monoclonal or polyclonal detection antibody reagent added. It is performed in the following four steps:

Separation of proteins by electrophoresis.

Immunofixation (immunoprecipitation) of the electrophoresed proteins—the appropriate electrophoretic migration tracks are overlaid with individual antisera. The antisera diffuse into the gel and precipitate the corresponding antigens when present. The proteins in the reference track are fixed with a fixative agent.

The unprecipitated, soluble proteins are removed from the gel by blotting and washing. Precipitation of the antigen-antibody complex is trapped within the gel matrix.

The precipitated proteins are visualized by staining (e.g. acid violet stain).

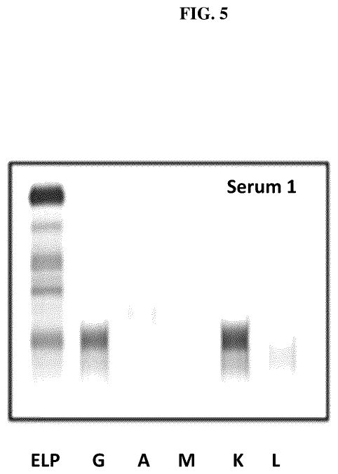

To detect and identify the suspected monoclonal component, the sample is simultaneously electrophoresed in several tracks in parallel (see FIG. 5). After the electrophoresis, the ELP track serves as a reference (containing the total protein fixation) providing a complete electrophoretic pattern of the serum sample's proteins. The remaining tracks allow characterization of the monoclonal component from its reaction, or lack of, usually with antisera against human IgG, IgA and IgM heavy chains, and against free and bound kappa and lambda light chains. Other anti-sera (e.g. anti-IgD, etc.) are also possible. The immunofixed bands are then compared with the suspect bands in the reference pattern-the corresponding band should have the same migration position.

FIG. 5 shows an example of a gel after serum immunofixation electrophoresis. A serum sample from a healthy donor was separated via gel electrophoresis 6 times in parallel whereas each lane was stained with a different reagent. After staining non complexed proteins were removed by blotting and washing. Lane ELP=total protein staining; Lane G=anti-IgG staining; Lane A=anti-IgA staining; Lane M=anti-IgM staining; Lane K=anti-kappa staining; Lane L=anti-lambda staining.

### WORKING EXAMPLES

### Materials and Method IFE

Immunofixation was performed using Sebia's semi-automated agarose gel electrophoresis systems Hydrasys and Hydrasys2 and using Sebia's Maxikit Hydragel 9IF. The kits are designed for detection of Immunoglobulins in human serum by immunofixation electrophoresis and contain all needed reagents and materials i.e. agarose gels, buffered strips, diluent, acid violet stain, antisera (e.g. IgG, IgA, IgM, Kappa and Lambda), fixative solution and applicators.

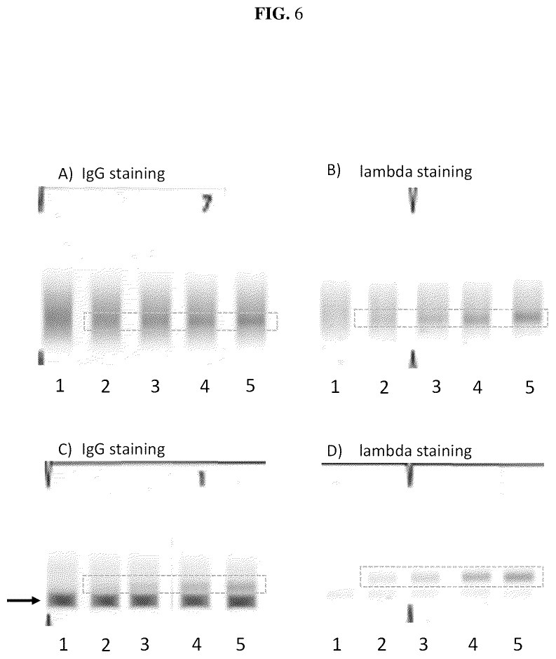

In order to evaluate the impact of MOR202 on M-protein analysis, serum samples from healthy donors and MM patients were spiked with MOR202 at different concentrations and incubated for at least 15 min at room temperature (RT). Afterwards the samples were analyzed either spiked or unspiked with MOR202 using IFE and gels were stained with anti-IgG or anti-lambda antisera (both staining reagents are able to bind to MOR202). In both stainings MOR202was detected already at the lowest concertation tested at 200 μg/mL suggesting IFE interference at or even below this drug serum level (FIG. 6).

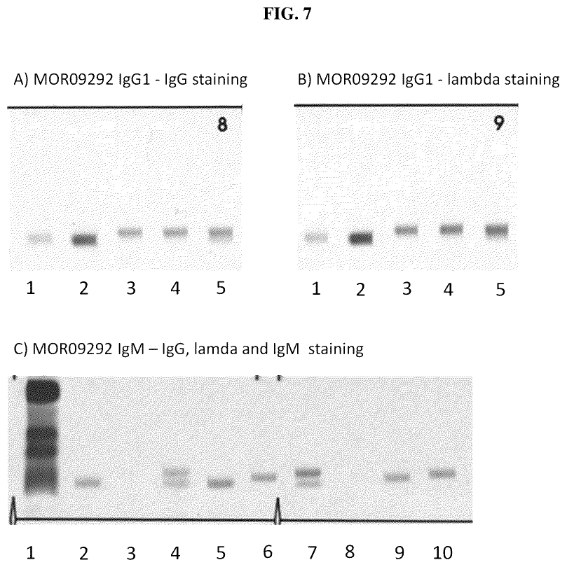

In order to differentiate between MOR202 related assay signals in IFE vs. endogenous M-protein spikes a method was tested pre-incubating a MOR202 containing sample with an MOR202 specific anti-idiotypic antibody (MOR09292). The objective of this method was to demonstrate that the MOR202 related IFE assay signal can be migrated comparing samples with or without pre-incubation of MOR09292 and therefore clearly identifying MOR202 related assay signals. For evaluating if the migration distance is large enough to be detected samples containing MOR202 in saline were prepared and pre-incubated with or without MOR09292. The anti-idiotypic antibody was produced and tested in an IgG1 as well as in IgM antibody format. Test samples were prepared at a constant concentration of 1200 μg/mL MOR202 and pre-incubated without or with various concentrations of the two MOR09292 variants for 60 min. Afterwards, the samples were analyzed and IFE gels were stained with anti-IgG or anti-lambda antisera. The results were that no acceptable migration distance of the MOR202 drug spike suitable for clinical sample assessment could be observed when the test samples were pre-incubated with the various forms of MOR09292 (FIG. 7). The surprising finding demonstrate that even when increasing the size of the drug/antibody complex compared to the drug antibody alone approximately 3-fold (MOR09292-IgG) or 7-fold (MOR09292-IgM) the change in molecular weight of the complex does not lead to a relevant shift in assay signals (i.e. changed migration pattern).

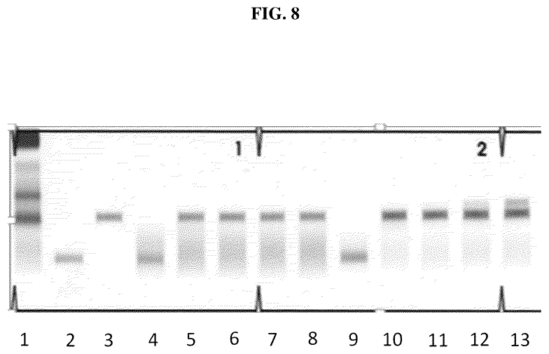

Based on these results a further variant of the idiotypic antibody was generated genetically fusing MOR09292-Fab fragment to human albumin (MOR09292-hAlb). The new variant increased the size of the drug-antibody complex compared to the drug antibody alone up to 2.6-fold. More important the incorporation of human serum albumin lowered the overall net-charge of the complex. Sample preparation and testing was performed as described above. As a result a clear shift of the MOR202/MOR09292-huAlb complex could be observed when compared to the assay signal of MOR202 alone, see FIG. 8.

The modified IFE assay using MOR09292-hAlb for sample pre-treatment was incorporated into the clinical development of MOR202. Therefore, the assay was validated at the central laboratory responsible for M-protein analysis and introduced into the testing strategy as “Immunofixation (IFE) Reflex Assay”. In order to discriminate between MOR202 and M-protein related signals the IFE Reflex Assay was performed in addition to the regular Serum IFE and Serum Protein Electrophoresis (SPE) for example when the following 2 conditions are fulfilled:
a) reduction in serum M-protein levels at least ≥40% compared to the M-protein concentration pre-treatment, and
b) at least one of the M-protein spikes left is identical to the characteristics of the drug antibody MOR202 (i.e. IgG/lambda positive staining in IFE).

### Case Study for the Use and Results of the IFE Reflex Assay Within Clinical Study MOR202C101

Within the first clinical study treating multiple myeloma patients with MOR202(MOR202C101) the IFE Reflex Assay was applied for patient 19007 after a reduction of ≥86% in serum M-protein levels was observed. For this patient, the M-protein spike identified was described by IFE as IgG/lambda positive, the same molecular properties as known for MOR202. Performing SPE a remaining concentration of potential M-proteins at 1 or 2 g/L was detected on Jan. 12, 2106 and Feb. 19, 2016. The IFE Reflex Assay could demonstrate that this assay signal was solely caused by MOR202 interference and therefore not M-protein related (see summary laboratory results Table 1). The results demonstrate how the newly established IFE Reflex Assay could clearly discriminate between M-protein and therefore disease related assay signals versus MOR202 treatment related assay signals.

TABLE 1
IFE Reflex Assay result negative

| | Sample reception date | | | | | | | | | | |
|---|---|---|---|---|---|---|---|---|---|---|---|
| | 19 Feb. | 11 Jan. | 14 Dec. | 16 Nov. | 26 Oct. | 21 Sep. | 7 Sep. | 24 Aug. | 10 Aug. | 27 Jul. | 17 Jul. |
| | 2016 | 2016 | 2015 | 2015 | 2015 | 2015 | 2015 | 2015 | 2015 | 2015 | 2015 |
| Visit | CYCLE 8/ | CYCLE 7/ | CYCLE 6/ | CYCLE 5/ | CYCLE 4/ | CYCLE 3/ | CYCLE 2/ | CYCLE 2/ | CYCLE 1/ | CYCLE 1/ | SCREENING |
| | DAY 1 | DAY 1 | DAY 1 | DAY 1 | DAY 8 | DAY 1 | DAY 15 | DAY 1 | DAY 15 | DAY 1 | |

SERUM PROTEIN ELECTROPHORESIS

| | | | | | | | | | | | |
|---|---|---|---|---|---|---|---|---|---|---|---|
| Monoclonal | 2 | 1 | 2 | 2 | 2 | 3 | 4 | 6 | 9 | 16 | 15 |
| peak 1 (Abs) | | | | | | | | | | | |
| Unit | g/L | g/L | g/L | g/L | g/L | g/L | g/L | g/L | g/L | g/L | g/L |

SERUM PROTEIN IMMUNOFIXATION

| | | | | | | | | | | | |
|---|---|---|---|---|---|---|---|---|---|---|---|
| Monoclonal | IgG/l | IgG/l | IgG/l | IgG/l | IgG/l | IgG/l | IgG/l | IgG/l | IgG/l | IgG/l | IgG/l |
| peak 1 | | | | | | | | | | | |

Immunofix, Reflex Assay

| | | | | | | | | | | | |
|---|---|---|---|---|---|---|---|---|---|---|---|
| Monoclonal | Negative | Negative | Positive | — | — | — | — | — | — | — | — |
| peak 1 | | | | | | | | | | | |

Table 1: Case study for the clinical use of the modified IFE assay applying a MOR09292-hAlb sample pre-treatment step—summary laboratory report patient 19007

Before starting treatment with MOR202 patient 19007 was tested positive for serum M-protein (16 g/L for sample received Jul. 27, 2016—positive staining for IgG/lambda in IFE). After a ≥86% reduction in serum M-protein levels was observed (14-Dec. 2015) the IFE Reflex assay was performed in addition to IFE and SPE. On Jan. 12, 2016 and Feb. 19, 2016 it was shown that the remaining M-protein concentration of 1 or 2 g/L, respectively, was only caused by MOR202 assay interference (i.e. Immunofixation Reflex Assay result “Negative” for M-proteins).

### EMBODIMENTS

An aspect is an anti-idiotypic antibody fused to albumin.

In an embodiment, albumin is human albumin having the amino acid sequence of SEQ ID NO: 6.

In an embodiment, the human albumin is a fragment of human albumin or partial sequence of human albumin.

In an embodiment, albumin is a functional fragment of albumin.

In another embodiment, human albumin is a functional fragment of human albumin. In this context the term “functional fragment” of albumin or human albumin refers to albumin which is a fragment or a variant of native albumin or human albumin, but which still is functional active in a sense that it is still able to fulfill the physiological role of albumin.

An embodiment, is an anti-idiotypic antibody that is specific for an antibody having a variable heavy domain comprising the amino acid sequence

(SEQ ID NO: 7)
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS,

and
a variable light chain domain comprising the amino acid sequence

(SEQ ID NO: 8)
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ.

An aspect, is an anti-idiotypic antibody that is specific for an antibody having a variable heavy domain comprising the amino acid sequence

(SEQ ID NO: 7)
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS,

and
a variable light chain domain comprising the amino acid sequence

(SEQ ID NO: 8)
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ.

In an embodiment the anti-idiotypic antibody is fused to albumin. In an embodiment, albumin is human albumin having the amino acid sequence of SEQ ID NO: 6. In an embodiment, the human albumin is a fragment of human albumin or partial sequence of human albumin. In an embodiment, the human albumin is a functional fragment of human albumin or partial sequence of human albumin.

In embodiments, the anti-idiotypic antibody comprises a variable heavy chain comprising an HCDR1 of amino acid sequence YSFSNYWIS (SEQ ID NO: 18), an HCDR2 of amino acid sequence WMGIIDPASSKTRYSPSFQG (SEQ ID NO: 19), an HCDR3 of amino acid sequence SRGAGMDY (SEQ ID NO: 20), and a variable light chain comprising an LCDR1of amino acid sequence TGSSSNIGAGYDVH (SEQ ID NO: 21), an LCDR2 of amino acid sequence LLIYADNNRPS (SEQ ID NO: 22), an LCDR3 of amino acid sequence GSYDESSNSM (SEQ ID NO: 23).

In an embodiment, the anti-idiotypic antibody is a human antibody.

In embodiments, the anti-idiotypic antibody comprises the variable heavy chain of amino acid sequence

(SEQ ID NO: 16)
QVQLVQSGAEVKKPGESLKISCKGSGYSFSNYWISWVRQMPGKGLEWMGI
IDPASSKTRYSPSFQGQVTISADKSISTAYLQWSSLKASDTAMYYCARSR
GAGMDYWGQGTLVTVSS,

and
   the variable light chain of amino acid sequence

(SEQ ID NO: 17)
DIVLTQPPSVSGAPGQRVTISCTGSSSNIGAGYDVHWYQQLPGTAPKLLI
YADNNRPSGVPDRFSGSKSGTSASLAITGLQSEDEADYYCGSYDESSNSM
VFGGGTKLTVLGQ.

In embodiments, the anti-idiotypic antibody albumin fusion comprises the heavy chain amino acid sequence

(SEQ ID NO: 24)
QVQLVQSGAEVKKPGESLKISCKGSGYSFSNYWISWVRQMPGKGLEWMGI
IDPASSKTRYSPSFQGQVTISADKSISTAYLQWSSLKASDTAMYYCARSR
GAGMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSDIDAHKSEVAHRFKDLGEENFKALVLIAFAQ
YLQQCPFEDHVKLVNEVTEFAKTCVADESAENCDKSLHTLFGDKLCTVAT
LRETYGEMADCCAKQEPERNECFLQHKDDNPNLPRLVRPEVDVMCTAFHD
NEETFLKKYLYEIARRHPYFYAPELLFFAKRYKAAFTECCQAADKAACLL
PKLDELRDEGKASSAKQRLKCASLQKFGERAFKAWAVARLSQRFPKAEFA
EVSKLVTDLTKVHTECCHGDLLECADDRADLAKYICENQDSISSKLKECC
EKPLLEKSHCIAEVENDEMPADLPSLAADFVESKDVCKNYAEAKDVFLGM
FLYEYARRHPDYSVVLLLRLAKTYETTLEKCCAAADPHECYAKVFDEFKP
LVEEPQNLIKQNCELFEQLGEYKFQNALLVRYTKKVPQVSTPTLVEVSRN
LGKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTAL
VELVKHKPKATKEQLKAVMDDFAAFVEKCCKADDKETCFAEEGKKLVAAS
QAALGLVNSRHHHHHH.

In embodiments, the anti-idiotypic antibody albumin fusion comprises the light chain amino acid sequence

(SEQ ID NO: 25)
DIVLTQPPSVSGAPGQRVTISCTGSSSNIGAGYDVHWYQQLPGTAPKLLI
YADNNRPSGVPDRFSGSKSGTSASLAITGLQSEDEADYYCGSYDESSNSM
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS.

An aspect is a method of evaluating a blood sample obtained from a patient undergoing treatment for multiple myeloma or other gammopathy comprising
a) obtaining a blood sample from said patient,
b) incubating the blood sample with an anti-idiotypic antibody,
c) performing immunofixation electrophoresis (IFE), and
d) reporting the results of the IFE.

In embodiments of the method, the patient is undergoing treatment with the antibody having a variable heavy domain comprising the amino acid sequence

(SEQ ID NO: 7)
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS,

and
a variable light chain domain comprising the amino acid sequence

(SEQ ID NO: 8)
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ.

In an embodiment of the method, the anti-idiotypic antibody is fused to albumin. In an embodiment of the method, albumin is human albumin having the amino acid sequence of SEQ ID NO: 6. In an embodiment of the method, the human albumin is a fragment of human albumin or partial sequence of human albumin.

The exemplified anti-idiotypic antibody MOR09292 is specific for MOR202. The anti-idiotypic antibody to MOR202, when fused to human albumin, shifted the antibody in IFE thus mitigating any potential interference of MOR202 with the M-protein based clinical assessment. It is expected that the fusion of other anti-idiotypic antibody that our specific for other antibodies that are used for therapy in multiple myeloma or other gammopathy would have a similar result. Meaning that other anti-idiotypic antibody albumin fusions would be useful in shifting the antibody in IFE thus mitigating any potential interference of that antibody with the M-protein based clinical assessment.

In embodiments of the method, the anti-idiotypic antibody comprises a variable heavy chain comprising
an HCDR1 of amino acid sequence YSFSNYWIS (SEQ ID NO: 18),
an HCDR2 of amino acid sequence WMGIIDPASSKTRYSPSFQG (SEQ ID NO: 19), an HCDR3 of amino acid sequence SRGAGMDY (SEQ ID NO: 20), and
a variable light chain comprising an LCDR1 of amino acid sequence TGSSSNIGAGYDVH (SEQ ID NO: 21), an LCDR2 of amino acid sequence LLIYADNNRPS (SEQ ID NO: 22), an LCDR3 of amino acid sequence GSYDESSNSM (SEQ ID NO: 23).

In embodiments of the method, the anti-idiotypic antibody is a human antibody.

In embodiments of the method, the anti-idiotypic antibody comprises the variable heavy chain of amino acid sequence

(SEQ ID NO: 16)
QVQLVQSGAEVKKPGESLKISCKGSGYSFSNYWISWVRQMPGKGLEWMGI
IDPASSKTRYSPSFQGQVTISADKSISTAYLQWSSLKASDTAMYYCARSR
GAGMDYWGQGTLVTVSS,

and
   the variable light chain of amino acid sequence

(SEQ ID NO: 17)
DIVLTQPPSVSGAPGQRVTISCTGSSSNIGAGYDVHWYQQLPGTAPKLLI
YADNNRPSGVPDRFSGSKSGTSASLAITGLQSEDEADYYCGSYDESSNSM
VFGGGTKLTVLGQ.

In embodiments of the method, the anti-idiotypic antibody albumin fusion comprises the heavy chain amino acid sequence

(SEQ ID NO: 24)
QVQLVQSGAEVKKPGESLKISCKGSGYSFSNYWISWVRQMPGKGLEWMGI
IDPASSKTRYSPSFQGQVTISADKSISTAYLQWSSLKASDTAMYYCARSR
GAGMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSDIDAHKSEVAHRFKDLGEENFKALVLIAFAQ
YLQQCPFEDHVKLVNEVTEFAKTCVADESAENCDKSLHTLFGDKLCTVAT
LRETYGEMADCCAKQEPERNECFLQHKDDNPNLPRLVRPEVDVMCTAFHD
NEETFLKKYLYEIARRHPYFYAPELLFFAKRYKAAFTECCQAADKAACLL
PKLDELRDEGKASSAKQRLKCASLQKFGERAFKAWAVARLSQRFPKAEFA
EVSKLVTDLTKVHTECCHGDLLECADDRADLAKYICENQDSISSKLKECC
EKPLLEKSHCIAEVENDEMPADLPSLAADFVESKDVCKNYAEAKDVFLGM
FLYEYARRHPDYSVVLLLRLAKTYETTLEKCCAAADPHECYAKVFDEFKP
LVEEPQNLIKQNCELFEQLGEYKFQNALLVRYTKKVPQVSTPTLVEVSRN
LGKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTAL
VELVKHKPKATKEQLKAVMDDFAAFVEKCCKADDKETCFAEEGKKLVAAS
QAALGLVNSRHHHHHH.

In embodiments of the method, the anti-idiotypic antibody albumin fusion comprises the light chain amino acid sequence

(SEQ ID NO: 25)
DIVLTQPPSVSGAPGQRVTISCTGSSSNIGAGYDVHWYQQLPGTAPKLLI
YADNNRPSGVPDRFSGSKSGTSASLAITGLQSEDEADYYCGSYDESSNSM
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS.

In embodiments of the method, the sample is obtained from a patient undergoing

treatment for multiple myeloma or other gammopathy. In further embodiments, the gammopathy is a monoclonal gammopathy. In further embodiments, the monoclonal gammopathies include: Hodgkin's disease; variants of multiple myeloma, e.g., Solitary plasmacytoma of bone, Extramedullary plasmacytoma, Plasma cell leukemia, and Non-secretory myeloma, Lymphoproliferative disorders, such as, Waldenström's macroglobulinemia, and Lymphoma; Heavy chain disease (γ, α, μ); and Amyloidosis.

In embodiments of the method, the sample is evaluated for total M-protein levels.

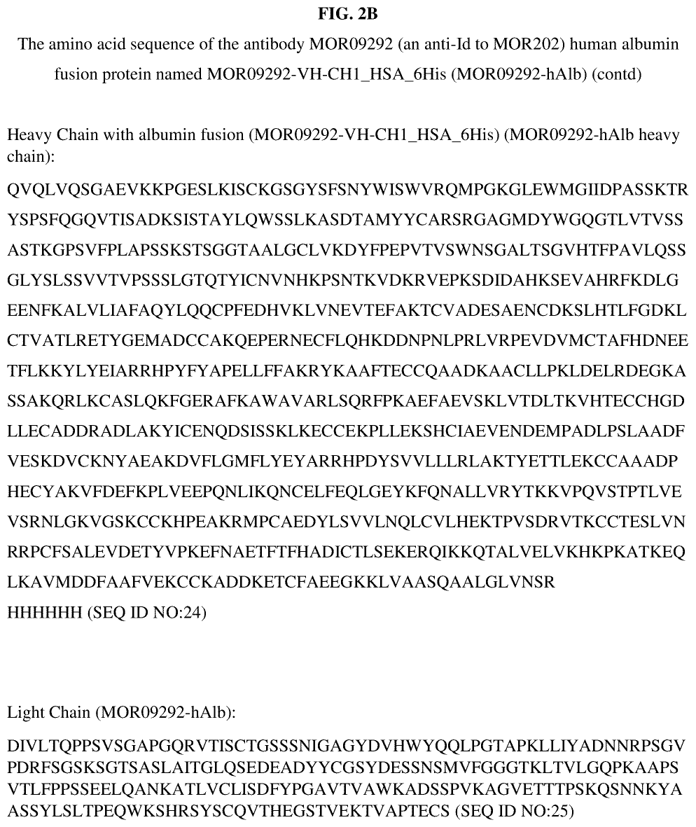

An aspect, is a nucleic acid encoding the exemplified anti-idiotypic antibody or anti-idiotypic antibody albumin fusion. In an embodiment, the anti-idiotypic antibody is MOR09292. In an embodiment the anti-idiotypic antibody is encoded by nucleic acid sequences encoding the amino acid sequences shown in FIGS. 2A-B.

In an embodiment the anti-idiotypic antibody is encoded by nucleic acid sequences SEQ ID NO: 26 (VH) and SEQ ID NO: 27 (VL).

1-12. (canceled)

13. A monoclonal anti-idiotypic antibody that is specific for an anti-CD38 antibody, wherein the anti-CD38 antibody comprises a variable heavy chain comprising the amino acid sequence of SEQ ID NO: 7, and a variable light chain comprising the amino acid sequence of SEQ ID NO: 8,
wherein the monoclonal anti-idiotypic antibody comprises a variable heavy chain comprising an HCDR1 comprising the amino acid sequence of SEQ ID NO: 18, an HCDR2 comprising the amino acid sequence of SEQ ID NO: 19, an HCDR3 comprising the amino acid sequence of SEQ ID NO: 20, and a variable light chain comprising an LCDR1 comprising the amino acid sequence of SEQ ID NO: 21, an LCDR2 comprising the amino acid sequence of SEQ ID NO: 22, an LCDR3 comprising the amino acid sequence of SEQ ID NO: 23.

14. The monoclonal anti-idiotypic antibody of claim 13, wherein the monoclonal anti-idiotypic antibody comprises a variable heavy chain comprising the amino acid sequence of SEQ ID NO: 16, and a variable light chain comprising the amino acid sequence of SEQ ID NO: 17.

15. The monoclonal anti-idiotypic antibody of claim 13, wherein the monoclonal anti-idiotypic antibody is an IgG.

16. The monoclonal anti-idiotypic antibody of claim 13, wherein the monoclonal anti-idiotypic antibody is an IgM.

17. A monoclonal anti-idiotypic antibody that is specific for an anti-CD38 antibody, wherein the anti-CD38 antibody comprises a variable heavy chain comprising the amino acid sequence of SEQ ID NO: 7, and a variable light chain comprising the amino acid sequence of SEQ ID NO: 8,
wherein the monoclonal anti-idiotypic antibody is not fused to albumin, and wherein the monoclonal anti-idiotypic antibody comprises a variable heavy chain comprising an HCDR1 comprising the amino acid sequence of SEQ ID NO: 18, an HCDR2 comprising the amino acid sequence of SEQ ID NO: 19, an HCDR3 comprising the amino acid sequence of SEQ ID NO: 20, and a variable light chain comprising an LCDR1 comprising the amino acid sequence of SEQ ID NO: 21, an LCDR2 comprising the amino acid sequence of SEQ ID NO: 22, an LCDR3 comprising the amino acid sequence of SEQ ID NO: 23.

18. A monoclonal anti-idiotypic antibody fused to albumin, wherein:
a) the monoclonal anti-idiotypic antibody is specific for a therapeutic antibody specific for CD38 , and
b) wherein the monoclonal anti-idiotypic antibody comprises a variable heavy chain comprising an HCDR1 comprising the amino acid sequence of SEQ ID NO: 18 and an HCDR2 comprising the amino acid sequence of SEQ ID NO: 19, and a variable light chain comprising an LCDR1 comprising the amino acid sequence of SEQ ID NO: 21 and an LCDR2 comprising the amino acid sequence of SEQ ID NO: 22.

19. The monoclonal anti-idiotypic antibody of claim 18, wherein the monoclonal anti-idiotypic antibody is an IgG1 isotype.

20. The monoclonal anti-idiotypic antibody of claim 18, wherein the monoclonal anti-idiotypic antibody is an IgM isotype.

21. The monoclonal anti-idiotypic antibody of claim 18, wherein the albumin is a functional fragment of human albumin.

22. The monoclonal anti-idiotypic antibody of claim 18, wherein the albumin is a human albumin having the amino acid sequence of SEQ ID NO: 6.

23. A method of evaluating a blood sample obtained from a subject comprising a) obtaining the blood sample from the subject, b) incubating the blood sample with the anti-idiotypic antibody of claim 13, and c) performing immunofixation electrophoresis (IFE).

24. The method of claim 23, further comprising d) evaluating the blood sample for total M-protein levels.

25. The method of claim 23, wherein the subject is undergoing treatment with the anti-CD38 antibody.

26. The method of claim 23, wherein the monoclonal anti-idiotypic antibody is fused to albumin.

27. The method of claim 26, wherein the albumin is human albumin having the amino acid sequence of SEQ ID NO: 6.

28. The method of claim 23, wherein the subject is undergoing treatment for multiple myeloma.

29. The method of claim 23, wherein the subject is undergoing treatment for a gammopathy.

30. The method of claim 29, wherein the gammopathy is selected from Hodgkin's disease, Waldenstrom's macroglobulinemia, heavy chain disease, and amyloidosis.

31. A nucleic acid encoding the monoclonal anti-idiotypic antibody of claim 13.

32. The nucleic acid of claim 31, wherein the nucleic acid comprises a sequence according to SEQ ID NO: 26 or SEQ ID NO: 27.


SEQUENCE LISTING (28 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - DNA, 360 bp, synthetic construct
  misc_feature 1..360: note = source = /note="Description of Artificial Sequence: Synthetic polynucleotide"
  source 1..360: mol_type = other DNA
caggtgcaattggtggaaagcggcggcggcctggtgcaaccgggcggcagcctgcgtctgagctgcgcggcctccggatttaccttttcttcttattatatgaattgggtgcgccaagcccctgggaagggtctcgagtgggtgagcggtatctctggtgatcctagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatcttcctcttgtttatactggttttgcttattggggccaaggcaccctggtgacggttagctca

SEQ ID NO: 3 - DNA, 327 bp, synthetic construct
  misc_feature 1..327: note = source = /note="Description of Artificial Sequence: Synthetic polynucleotide"
  source 1..327: mol_type = other DNA
gatatcgaactgacccagccgccttcagtgagcgttgcaccaggtcagaccgcgcgtatctcgtgtagcggcgataatcttcgtcattattatgtttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgccagacttatactggtggtgcttctcttgtgtttggcggcggcacgaagttaaccgttcttggccag

SEQ ID NO: 4 - DNA, 351 bp, synthetic construct
  misc_feature 1..351: note = source = /note="Description of Artificial Sequence: Synthetic polynucleotide"
  source 1..351: mol_type = other DNA
caggtgcaattggttcagagcggcgcggaagtgaaaaaaccgggcgaaagcctgaaaattagctgcaaaggttccggatattccttttctaattattggatttcttgggtgcgccagatgcctgggaagggtctcgagtggatgggcattatcgatccggcttctagcaagacccgttattctccgagctttcagggccaggtgaccattagcgcggataaaagcattagcaccgcgtatcttcaatggagcagcctgaaagcgagcgatacggccatgtattattgcgcgcgttctcgtggtgctggtatggattattggggccaaggcaccctggtgacggttagctca

SEQ ID NO: 5 - DNA, 339 bp, synthetic construct
  misc_feature 1..339: note = source = /note="Description of Artificial Sequence: Synthetic polynucleotide"
  source 1..339: mol_type = other DNA
gatatcgtgctgacccagccgccttcagtgagtggcgcaccaggtcagcgtgtgaccatctcgtgtacgggcagcagcagcaacattggtgctggttatgatgtgcattggtaccagcagttgcccgggacggcgccgaaacttctgatttatgctgataataatcgtccctcaggcgtgccggatcgttttagcggatccaaaagcggcaccagcgcgagccttgcgattacgggcctgcaaagcgaagacgaagcggattattattgcggttcttatgatgagtcttctaattctatggtgtttggcggcggcacgaagttaaccgttcttggccag

SEQ ID NO: 6 - protein, 609 aa, Homo sapiens [sequence omitted]

SEQ ID NO: 7 - protein, 120 aa, synthetic construct
  REGION 1..120: note = source = /note="Description of Artificial Sequence: Synthetic polypeptide"
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 8 - protein, 109 aa, synthetic construct
  REGION 1..109: note = source = /note="Description of Artificial Sequence: Synthetic polypeptide"
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 9 - protein, 10 aa, synthetic construct
  REGION 1..10: note = source = /note="Description of Artificial Sequence: Synthetic peptide"
GFTFSSYYMN

SEQ ID NO: 10 - protein, 5 aa, synthetic construct
  REGION 1..5: note = source = /note="Description of Artificial Sequence: Synthetic peptide"
SYYMN

SEQ ID NO: 11 - protein, 17 aa, synthetic construct
  REGION 1..17: note = source = /note="Description of Artificial Sequence: Synthetic peptide"
GISGDPSNTYYADSVKG

SEQ ID NO: 12 - protein, 11 aa, synthetic construct
  REGION 1..11: note = source = /note="Description of Artificial Sequence: Synthetic peptide"
DLPLVYTGFAY

SEQ ID NO: 13 - protein, 11 aa, synthetic construct
  REGION 1..11: note = source = /note="Description of Artificial Sequence: Synthetic peptide"
SGDNLRHYYVY

SEQ ID NO: 14 - protein, 7 aa, synthetic construct
  REGION 1..7: note = source = /note="Description of Artificial Sequence: Synthetic peptide"
GDSKRPS

SEQ ID NO: 15 - protein, 8 aa, synthetic construct
  REGION 1..8: note = source = /note="Description of Artificial Sequence: Synthetic peptide"
QTYTGGAS

SEQ ID NO: 16 - protein, 117 aa, synthetic construct
  REGION 1..117: note = source = /note="Description of Artificial Sequence: Synthetic polypeptide"
QVQLVQSGAEVKKPGESLKISCKGSGYSFSNYWISWVRQMPGKGLEWMGIIDPASSKTRYSPSFQGQVTISADKSISTAYLQWSSLKASDTAMYYCARSRGAGMDYWGQGTLVTVSS

SEQ ID NO: 17 - protein, 113 aa, synthetic construct
  REGION 1..113: note = source = /note="Description of Artificial Sequence: Synthetic polypeptide"
DIVLTQPPSVSGAPGQRVTISCTGSSSNIGAGYDVHWYQQLPGTAPKLLIYADNNRPSGVPDRFSGSKSGTSASLAITGLQSEDEADYYCGSYDESSNSMVFGGGTKLTVLGQ

SEQ ID NO: 18 - protein, 9 aa, synthetic construct
  REGION 1..9: note = source = /note="Description of Artificial Sequence: Synthetic peptide"
YSFSNYWIS

SEQ ID NO: 19 - protein, 20 aa, synthetic construct
  REGION 1..20: note = source = /note="Description of Artificial Sequence: Synthetic peptide"
WMGIIDPASSKTRYSPSFQG

SEQ ID NO: 20 - protein, 8 aa, synthetic construct
  REGION 1..8: note = source = /note="Description of Artificial Sequence: Synthetic peptide"
SRGAGMDY

SEQ ID NO: 21 - protein, 14 aa, synthetic construct
  REGION 1..14: note = source = /note="Description of Artificial Sequence: Synthetic peptide"
TGSSSNIGAGYDVH

SEQ ID NO: 22 - protein, 11 aa, synthetic construct
  REGION 1..11: note = source = /note="Description of Artificial Sequence: Synthetic peptide"
LLIYADNNRPS

SEQ ID NO: 23 - protein, 10 aa, synthetic construct
  REGION 1..10: note = source = /note="Description of Artificial Sequence: Synthetic peptide"
GSYDESSNSM

SEQ ID NO: 24 - protein, 816 aa, synthetic construct
  REGION 1..816: note = source = /note="Description of Artificial Sequence: Synthetic polypeptide"
QVQLVQSGAEVKKPGESLKISCKGSGYSFSNYWISWVRQMPGKGLEWMGIIDPASSKTRYSPSFQGQVTISADKSISTAYLQWSSLKASDTAMYYCARSRGAGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSDIDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVNSRHHHHHH

SEQ ID NO: 25 - protein, 217 aa, synthetic construct
  REGION 1..217: note = source = /note="Description of Artificial Sequence: Synthetic polypeptide"
DIVLTQPPSVSGAPGQRVTISCTGSSSNIGAGYDVHWYQQLPGTAPKLLIYADNNRPSGVPDRFSGSKSGTSASLAITGLQSEDEADYYCGSYDESSNSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - DNA, 2448 bp, synthetic construct
  misc_feature 1..2448: note = source = /note="Description of Artificial Sequence: Synthetic polynucleotide"
  source 1..2448: mol_type = other DNA
caggtgcaattggttcagagcggcgcggaagtgaaaaaaccgggcgaaagcctgaaaattagctgcaaaggttccggatattccttttctaattattggatttcttgggtgcgccagatgcctgggaagggtctcgagtggatgggcattatcgatccggcttctagcaagacccgttattctccgagctttcagggccaggtgaccattagcgcggataaaagcattagcaccgcgtatcttcaatggagcagcctgaaagcgagcgatacggccatgtattattgcgcgcgttctcgtggtgctggtatggattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatctgatatcgacgcccacaagagcgaggtggcccaccggtttaaggacctgggcgaggaaaacttcaaggccctggtgctgatcgccttcgcccagtacctgcagcagtgccccttcgaggaccacgtgaagctcgtgaacgaagtgaccgagttcgccaagacctgcgtggccgatgagagcgccgagaactgcgacaagagcctgcacaccctgttcggcgacaagctgtgtaccgtggccaccctgagagaaacctacggcgagatggccgactgctgcgccaagcaggaacccgagaggaacgagtgcttcctgcagcacaaggacgacaaccccaacctgcccagactcgtgcggcccgaagtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgttgccaggccgccgataaggccgcttgcctgctgcctaagctggacgagctgagggatgagggcaaggccagctctgccaagcagagactgaagtgcgccagcctgcagaagttcggcgagcgggcctttaaagcctgggccgtggctagactgagccagagattccccaaggccgagtttgccgaggtgtccaagctcgtgaccgacctgaccaaggtgcacaccgagtgctgtcacggcgacctgctggaatgcgccgacgacagagccgatctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgacttcgtggaaagcaaggacgtgtgcaagaactacgccgaggccaaggatgtgttcctgggcatgtttctgtatgagtacgcccgcagacaccccgactacagcgtggtgctgctgctgagactggccaaaacctacgagacaaccctggaaaagtgctgtgccgccgctgacccccacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagagaatgccctgcgccgaggactacctgtctgtggtgctgaaccagctgtgcgtgctgcacgagaaaacccccgtgtccgacagagtgaccaagtgctgtaccgagagcctcgtgaacagacggccctgcttcagcgccctggaagtggatgagacatacgtgcccaaagagttcaacgccgagacattcaccttccacgccgacatctgcaccctgtccgagaaagagcggcagatcaagaaacagaccgctctggtggaactcgtgaagcacaagcccaaggccaccaaagaacagctgaaggccgtgatggacgacttcgccgcctttgtggaaaaatgctgcaaggccgatgacaaagagacatgcttcgccgaagagggcaagaaactggtggccgcctctcaggctgctctgggactggttaactctagacaccatcaccatcaccat

SEQ ID NO: 27 - DNA, 651 bp, synthetic construct
  misc_feature 1..651: note = source = /note="Description of Artificial Sequence: Synthetic polynucleotide"
  source 1..651: mol_type = other DNA
gatatcgtgctgacccagccgccttcagtgagtggcgcaccaggtcagcgtgtgaccatctcgtgtacgggcagcagcagcaacattggtgctggttatgatgtgcattggtaccagcagttgcccgggacggcgccgaaacttctgatttatgctgataataatcgtccctcaggcgtgccggatcgttttagcggatccaaaagcggcaccagcgcgagccttgcgattacgggcctgcaaagcgaagacgaagcggattattattgcggttcttatgatgagtcttctaattctatggtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 28 - protein, 6 aa, synthetic construct
  REGION 1..6: note = source = /note="Description of Artificial Sequence: Synthetic 6xHis tag"
HHHHHH